(19) United States
(12) Patent Application Publication
(10) Pub. No.: US 2021/0079388 A1
(43) Pub. Date: Mar. 18, 2021

(54) SCREENING AND APPLICATION OF SGRNA FOR AHI1 GENE EDITING

(71) Applicant: SOOCHOW UNIVERSITY, Suzhou (CN)

(72) Inventors: Wanping SUN, Suzhou (CN); Bangsheng XI, Suzhou (CN)

(21) Appl. No.: 17/103,637

(22) Filed: Nov. 24, 2020

Related U.S. Application Data
(63) Continuation of application No. PCT/CN2018/091170, filed on Jun. 13, 2018.

(30) Foreign Application Priority Data
May 24, 2018 (CN) 201810510719.8

(51) Int. Cl. C12N 15/11; C12N 9/22; A61K 38/46; A61K 31/7088

(52) U.S. Cl. CPC C12N 15/11; C12N 9/22; C12N 2800/80; A61K 31/7088; C12N 2310/20; A61K 38/465

(57) ABSTRACT

An sgRNA for an AHI1 gene is provided. This Prokernel evaluation system confirms the editing efficiency of the sequence above with the experiment of white to blue clone formation. It is proved that the sequence has excellent editing efficiency. It also provides reference for AHI1 gene therapy in the future.

This is a Continuation of PCT/CN2018/091170, filed on Jun. 13, 2018, which claims priority to Chinese Application No. 201810510719.8, filed on May 24, 2018, which is incorporated by reference for all purposes as if fully set forth herein.

### TECHNICAL FIELD

The present invention belonged to gene technology, and particularly related to an sgRNA for AHI1 gene, which had high editing efficiency.

### BACKGROUND TECHNOLOGY

AHI1 (Abelson Helper Integration Site 1) gene could promote the development of human cerebellum and cortex. If a gene mutation occurs, Joubert syndrome could occur. The gene editing system of CRISPR/Cas9 (Clustered Regularly Interspaced Short Palindromic Repeats/Cas9) is the third-generation gene editing system developed from ZFNs and TALENs. It is discovered from the adaptive immune defense system of bacteria and used to combat foreign DNA. As well as invasive viruses, they have been widely used in the field of biomedicine.

### SUMMARY OF INVENTION

### Technical Problem

For specific target genes, a large number of sgRNA sequences could be designed because Cas9 enzyme could cut any target sequence adjacent to the PAM site, but the editing efficiency of each sgRNA is different. For example, the PAM site is 5′-NGG-3′ editing efficiency is usually higher than 5′-NGA-3′ or 5′-NAG-3′. The efficiency of gene editing in the CRISPR/Cas9 system is affected by many factors, among which the specificity between different sgRNA sequences is particularly important.

### Problem Solution

### Technical Solutions

The present invention disclosed the most optimal sgRNA of the AHI1 gene, which provided a reference for future gene therapy.

The present invention adopted the following technical solutions:

An sgRNA for AHI1 gene, the sequence of the sgRNA for AHI1 gene is SEQ ID NO: 1.

A drug of the AHI1 gene includes the sgRNA of SEQ ID NO: 1.

In the technical solution above, the drug of the AHI1 gene also included drug carriers, such as normal polymer-supports, cell carriers and so on.

A plasmid of the AHI1 gene includes a carrier and the sgRNA of SEQ ID NO: 1.

Preferably, the carriers were the pCas9 plasmid in the plasmid of the AHI1 gene.

Prokernel evaluation system confirmed the editing efficiency of the sequence above was 60% to 62%, with the experiment of white to blue clone formation in this invention.

Thereupon, this invention also disclosed the sequence was SEQ ID NO: 1 of the sgRNA, which was applicated in the preparation of the drug for AHI1 gene and Joubert syndrome.

First, a partial sequence of coding area β-gal of pMD-19T plasmid was replaced by a long sequence contained the target sequence to form a frameshift mutation. The replaced plasmid was called the pMD-repeat plasmid and was individually transformed into the solid medium with X-gal and IPTG couldn't form the blue colonies. When the co-transformed with pCas9 plasmid loaded with the target sequence of the corresponding sgRNA, the target sequence in the pMD-repeat plasmid was digested with Cas9 protease guided by sgRNA, and the before and after two repeat paragraphs of the target sequence would homologous recombination. So that the gene sequence of the encoding β-gal was restored from frame-shifting to non-frame-shifting state. Under the induction of X-gal and IPTG, blue colonies were formed.

### The Beneficial Effects of the Invention

### Beneficial Effect

A prokaryotic gene knockout pCas9 plasmid of the sgRNA sequence and a pMD-repeat plasmid contained the corresponding target sequence were constructed. The two plasmids were co-transformed into DH5a competent cells in equal amounts. X-gal-TPTG-chloramphenicol-ampicillin Culture, observe the proportion of blue colonies in the total colonies. Construct eukaryotic gene knockout pSpCas9 (BB)-2A-GFP plasmid contained sgRNA sequence, transfect HeLa cells, and perform gene editing. Then extract the cell genome from the control and experimental groups, and design primers in the upstream and downstream regions of the sgRNA. Q5 high-fidelity enzyme PCR, product purification, T7E1 enzyme digestion, agarose gel electrophoresis, and observation of band results after electrophoresis; design sequencing primers, sequence the purified product, and observe whether there are nested peaks and nested peaks near the Cas9 digestion site High and low. The data proves that the present invention discloses that the sgRNA targeting the AHI1 gene has optimal editing efficiency. Brief description of the drawings.

### BRIEF DESCRIPTION OF THE DRAWINGS

FIG. 1 is the schematic diagram of the sgRNA clone of the pCas9 plasmid;

FIG. 2 is the partial base sequence diagram of the lacZ gene of the pMD-repeat plasmid;

FIG. 3 is the plasmid diagram of pSpCas9 (BB)-2A-GFP;

FIG. 4 is the schematic diagram of the experimental principle of the formation of white-blue clone;

FIG. 5 is the colony diagram of AHI1-sgRNA dual plasmid co-transformation colony formation experiment of white blue clone;

FIG. 6 shows the ratio of blue colonies to total colonies of AHI1-sgRNA dual plasmid co-transformation experiments in the white to blue clone formation experiment (n=3, mean SD);

FIG. 7 is the diagram of pMD-repeat plasmid repair sequencing.

### INVENTION EXAMPLES

### Embodiments of the Invention

The reagents are all commercially available products.

### EXAMPLES

For the sgRNA of the AHI1 gene, the sequence of the sgRNA of the AHI1 gene is SEQ ID NO: 1. Specifically, 5′-GATAATGTCTCCGCGATGGATGG-3′.

### Comparative Example

The other three sgRNAs were selected for comparison, as follows:

SEQ ID NO: 2: 5′-CTCGGATAATGTCTCCGCGATGG-3′
SEQ ID NO: 3: 5′-AATTGGATATCCATCCCGGCTGG-3′
SEQ ID NO: 4: 5′-GATGAACTAACCATCCATCGCGG-3′

1. Constructed according to the conventional method and loaded with the 4 AHI1 above

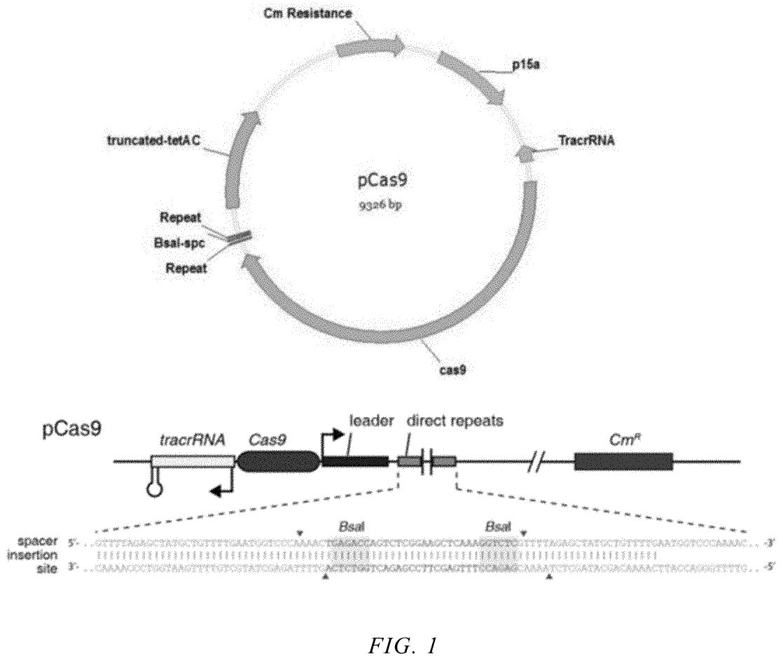

Prokaryotic gene of sgRNA knockout CRISPR/Cas9 plasmid, was shown by FIG. 1 for details; pCas9 plasmid digestion system (pCas9 plasmid 2 g, Bsal enzyme (NEB) 2 μL, 100×BSA (NEB) 1 μL, 10×NEB Buffer 10 μL, ddH₂O up to 100 μL), at 37° C. with water bath digestion overnight. The digested product and undigested plasmid were identified by agarose gel electrophoresis at the same time. After cutted, it was added to a 1.2% agarose gel well. With 120V electrophoresis about 30 min. As a reference with the 1 kb DNA Marker, the gel is purified and recovered. The experimental steps for purification and recovery are as follows:

(1) Added 500 μL of equilibrium solution BL to the adsorption column, centrifuge at 12000 rpm for 1 min, discard the waste liquid, and reposition the adsorption column in the collection tube; (2) Cut the desired band from the gel, and possibly remove excess gel, and weigh the cut gel;

(3) Added an equal volume of PC to the gel based on the gel weight (if the gel is 0.1 g, the volume is considered as 100 μL, the added PC volume is 100 μL), with 56° C. water bath for 10 min, and continuously invert during mixing;

(4) the dissolved liquid is cooled to room temperature, sucked into an adsorption column, centrifuged at 12000 rpm for 1 min, discarded the waste liquid is repositioned in the collection tube;

(5) Added 600 μL of PW to the adsorption column (anhydrous ethanol has been added to the rinsing solution PW) and let it stand for 2-4 min.

Centrifuge at 12000 rpm for 1 min, discard the waste liquid, and reposition the adsorption column in the collection tube. Repeat this operation once.

(6) Empty the collection tube contained the adsorption column at 12000 rpm for 2 min, and dry at room temperature;

(7) Took a new centrifuge tube, placed the adsorption column, and sucked 50 μL ddH₂O placed it in an adsorption column for 2 minutes and centrifuged at 12000 rpm for 2 minutes. The resulting solution is the purified plasmid digested product.

2. Phosphorylation of Sequence of the sgRNA

Dilute Oligo synthesized by Jinweizhi Company to 10 M, phosphorylated, the sequence of oligo sgRNA was as follows:

(SEQ ID NO: 5)
5′-AAACCTCGGATAATGTCTCCGCGAG-3′;
(SEQ ID NO: 6)
5′-AAAACTCGCGGAGACATTATCCGAG-3′;
(SEQ ID NO: 7)
5′-AAACGATAATGTCTCCGCGATGGAG-3′;
(SEQ ID NO: 8)
5′-AAAACTCCATCGCGGAGACATATTC-3′;
(SEQ ID NO: 9)
5′- AAACAATTGGATATCCATCCCGGCG-3′:
(SEQ ID NO: 10)
5′- AAAACGCCGGGATGGATATCCAATT-3′;
(SEQ ID NO: 11)
5′- AAACGATGAACTAACCATCCATCG-3′;
(SEQ ID NO: 12)
5′-AAAACGATGGATGGTTAGTTCATC-3′.

The above pairs correspond to SEQ ID NO: 2, SEQ ID NO: 1, SEQ ID NO: 3, and SEQ ID NO: 4, respectively.

The phosphorylation system was shown in Table 1, at 37° C. for 30 min.

TABLE 1
Phosphorylation system

| Components | volume | |
|---|---|---|
| Oligo F | 10 | μL |
| Oligo R | 10 | μL |
| T4 PNK enzyme | 1 | μL |
| 10X T4 Ligase Buffer | 5 | μL |

| | |
|---|---|
| ddH₂O | up to 50 μL |

sgRNA annealed, added 2.5 μL.

1 M sodium chloride into the phosphorylated product, annealed with a PCR machine for 2 h, slowly cooled from 95° C. to room temperature, and the final product was diluted 10 times.

3. The connection system was shown in Table 2. The system was reacted at 16° C. overnight.

TABLE 2
Ligation reaction system

| Components | volume |
|---|---|
| Oligo products after annealing | 2 μL |
| pCas9 plasmid carrier after enzyme digestion | 1 μL |
| T4 Ligase (NEB) | 1 μL |
| 10X T4 Ligase Buffer (NEB) | 2 μL |
| ddH₂O | up to 20 μL |

4. Conversion

(1) Take 50 μL DH5α competent cells and placed them on ice, add the product at 20° C. after the above connection, gently mix and let stand for 30 min;

(2) Heat for 45 seconds in a 42° C. water bath, and quickly placed it in ice for 10 minutes;

(3) Added antibiotic-free LB liquid culture medium 800 μL, 37° C., 220 rpm constant temperature shaking culture for 50 min.

(4) In a clean bench, transfer the bacterial solution to a chloramphenicol-contained LB solid medium with a pipette. On the plate, let stand for 20 min, and invert in an incubator with 37° C.;

(5) After 12 h, 10 colonies were picked and cultured in LB liquid medium contained chloramphenicol, and the plasmid was extracted.

5. Extraction of Plasmid DNA

Follow the operating instructions of Tiangen's Plasmid Miniprep Kit (Cargo #DP103-03):

(1) Added 500 μL of equilibrium solution BL to the adsorption column, centrifuge at 12000 rpm for 1 min, discard the waste liquid, and reposition the adsorption column in the collection tube;

(2) 5 mL of the bacterial solution cultured overnight, centrifuged at 12000 rpm for 15 min, and discarded the supernatant;

(3) Added 250 μL solution P1 (included with the kit, RNaseA has been added) to a centrifuge tube contained bacterial cell pellet, vortex to dissolve the pellet;

(4) Added 250 μL solution P2 (supplied with the kit), and mix gently by inverting 8-10 times, so that the bacterial cells are cracked;

(5) Added 350 μL solution P3 (included in the kit) immediately and mix gently upside down 10 times. At this time, a white flocculent precipitate appears, and centrifuged at 12000 rpm for 10 minutes;

(6) Transfer the supernatant in the centrifuge tube to the adsorption column with a pipette, centrifuge at 12,000 rpm for 1 min, discard the waste liquid, and reposition the adsorption column in the collection tube;

(7) Added 600 μL to the adsorption column PW (anhydrous ethanol has been added to the rinsing solution PW), stand still for 3 min, centrifuge at 12000 rpm for 1 min, discard the waste liquid, and reposition the adsorption column in the collection tube, repeat this operation once;

(8) Empty the collection tube contained the adsorption column at 12000 rpm for 2 min, and dry at room temperature;

(9) Took a new centrifuge tube, placed the adsorption column, pipet 10 (VL ddH₂O) into the adsorption column, placed it for 2 min, centrifuge at 12000 rpm for 2 min, and measure its plasmid concentration and purity with Nanodrop 2000.

Appraisal

The extracted plasmid was sent to Suzhou Jinweizhi Company for sequencing, and it was checked whether the target fragment was inserted into the plasmid correctly and kept for future use.

6. Recombinant Plasmid-Target Sequence of pMD-Repeat Construction

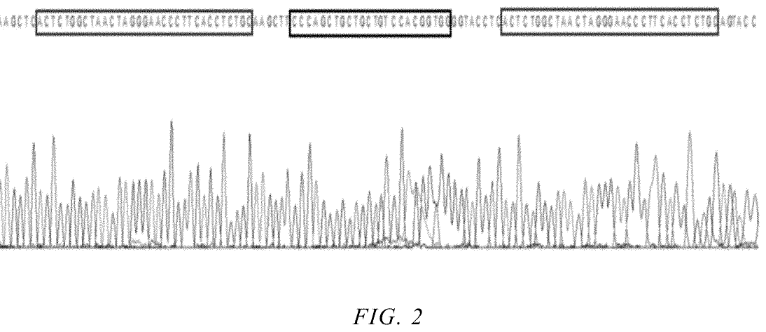

The pMD-repeat plasmid was modified from the pMD-19T plasmid. Using the HIV partial sequence as a reference, a long sequence was designed to replace the original sequence between the Kpnl and HindIII digestion sites in the lacZ gene of the PMD-19T plasmid. The long sequence contains two HIV repeated and one target sequence. The mutation of the original Kpnl and HindII restriction sited on the plasmid disappeared. The Kpnl and HindLII restriction sited between the two repeats and the target sequence could be used to insert the target sequence corresponding to the sgRNA. The read frame of the plasmid lacZ gene was frame-shifted after the modification, which generates a stop codon and cannot form α-complement. It was called pMD-repeat plasmid. Part of the base sequence of the lacZ gene of the pMD-repeat plasmid as FIG. 2, the red box represents the HIV repeat sequence; the black box represents the target sequence, and between the red box and the black box are the Kpnl and HindIII digestion sites.

7. pMD-Repeat Plasmid Digestion, Gel Purification and Recovery

The long sequence contained Kpnl and HindIII digestion sites in the pMD-repeat plasmid. Between the repeat sequence and the target sequence, the target sequence could be inserted after digestion. The pMD-repeat plasmid was digested with Kpnl and HindIII, and the digestion system (PMD-repeat plasmid 1 μg, Hind III enzyme 0.5 μL, Kpnl enzyme 0.5 μL, 1 OX NEB Buffer 2.1 2 μL, ddH₂O to 20 L), with 37° C. water bath reaction for 2 h; after digestion, the product was identified by agarose gel electrophoresis, and the gel was purified and recovered.

Oligonucleotide Complementary to sgRNA Target Sequence

The target oligonucleotides corresponding to the four AHI1 sgRNAs synthesized by Suzhou Jinweizhi Company were complementary. The reaction system is: 10× Anneal Buffer 2 μL, Oligo F 1 μL (10 μM), Oligo R 1 μL (10 μM), plus ddH₂O 16 μL, total volume 20 μL. The reaction conditions were: 95° C., 2 min; 1° C. per 30 sec to 65° C.; 65° C., 5 min; 1° C. per 1 min to 25° C.; 25° C., 1 min, and then cooled to 4° C. The target sequence of the oligonucleotide sequence was as follows:

(SEQ ID NO: 13)
AGCTTACTCGGATAATGTCTCCGCGATGGGGTAC;
(SEQ ID NO: 14)
CCCATCGCGGAGACATTATCCGAGTA;
(SEQ ID NO: 15)
AGCTTGGATAATGTCTCCGCGATGGATGGGGTAC;
(SEQ ID NO: 16)
CCCATCCATCGCGGAGACATCATTCCA;
(SEQ ID NO: 17)
AGCTTTAATTGGATATCCATCCCGGCTGGGGTAC;
(SEQ ID NO: 18)
CCCAGCCGGGATGGATATCCAATTAA;
(SEQ ID NO: 19)
AGCTTAGATGAACTAACCATCCATCGCGGGGTAC;
(SEQ ID NO: 20)
CCCGCGATGGATGGTTAGTTCATCTA.

The above pairs correspond to SEQ ID NO: 2, SEQ ID NO: 1, SEQ ID NO: 3, and SEQ ID NO: 4, respectively.

The purified and recovered pMD-repeat plasmid was ligated with the annealed complementary oligonucleotide strand. The system was digested with pMD-repeat plasmid 1 μL and annealed Oligo 7.5μ, T4 ligase (NEB) 0.5 μL, 10×T4 Buffer 1 μL, at 16° C. overnight reaction.

Transform the ligated product into DH5α competent cells, add 80 (VL LB liquid medium, 37° C., 40 min shaking culture, culture on a plate contained ampicillin resistance at 37° C., and after 12 h, pick 5 Each colony was cultured in LB liquid medium contained ampicillin, and the plasmid was extracted; the extracted plasmid was sent to Suzhou Jinweizhi Company for sequencing, and it was checked whether the target sequence was inserted into the plasmid correctly and stored for future use.

8. Construction of Recombinant pSpCas9 (BB)-2A-GFP Plasmid-sgRNA

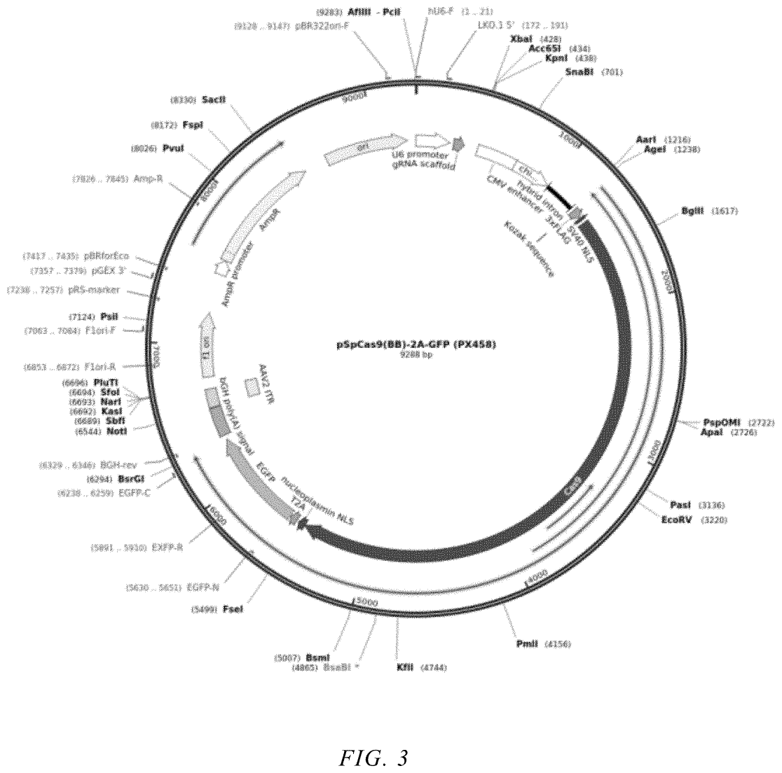

Construct four eukaryotic gene knockout CRISPR/Cas9 plasmids loaded with AHI1 sgRNA, was shown in FIG. 3.

pSpCas9 (BB)-2A-GFP plasmid digestion, gel purification and recovery, pSpCas9 (BB)-2A-GFP plasmid digestion system (pSpCas9 (BB)-2A-GFP plasmid 2 g, Bbsl enzyme (NEB) 2 μL, 100×BSA (NEB) 1 μL, 10×NEB Buffer 2.110 μL, ddH₂O up to 100 μL), with 37° C. water bath reaction for 4 h; digested product was identified by agarose gel electrophoresis, purified by gel digestion and recovered.

Dilute Oligo synthesized by Jinweizhi Company to 10 μM, phosphorylated, the sequence of Oligo sgRNA was as follows:

(SEQ ID NO: 21)
CACCGCTCGGATAATGTCTCCGCGA;
(SEQ ID NO: 22)
AAACTCGCGGAGACATTATCCGAGC;
(SEQ ID NO: 23)
CACCGATAATGTCTCCGCGATGGA;
(SEQ ID NO: 24)
AAACTCCATCGCGGAGACATTATC;
(SEQ ID NO: 25)
CACCGAATTGGATATCCATCCCGGC;
(SEQ ID NO: 26)
AAACGCCGGGATGGATATCCAATTC;
(SEQ ID NO: 27)
CACCGATGAACTAACCATCCATCG;
(SEQ ID NO: 28)
AAACCGATGGATGGTTAGTTCATC.

The above pairs correspond to SEQ ID NO: 2, SEQ ID NO: 1, SEQ ID NO: 3, and SEQ ID NO: 4, respectively.

The phosphorylation system was shown in Table 3, at 37° C. for 30 min. The phosphorylated product was annealed with a PCR machine for 2 h, 95° C., 5 min; 1° C. to 25° C. per 1 min; 25° C., 1 min, and then cooled to 4° C. Cool slowly to room temperature, dilute the final product 10-fold; then connect and connect the system as shown in Table 4, and react at 16° C. overnight; transform the connected product into DH5α competent cells, added 800 μL LB liquid medium, at 37° C. for 40 min shaked culture, cultured at 37° C. on a plate contained ampicillin resistance, and after 12-14 h, pick 3-5 colonies and cultured in LB liquid medium contained ampicillin, extracted the plasmid, and extract the plasmid. Send it to Suzhou Jinweizhi Company for sequencing, checked whether the target sequence was correctly inserted into the plasmid, and save it for future use.

TABLE 3
Phosphorylation system

| Components | volume | |
|---|---|---|
| Oligo F | 10 | μL |
| Oligo R | 10 | μL |
| T4 PNK enzyme | 1 | μL |
| 10X T4 Ligase Buffer | 5 | μL |

| | |
|---|---|
| ddH₂O | up to 50 μL |

TABLE 4
Ligation reaction system

| Components | volume |
|---|---|
| Oligo products after annealing | 2 μL |
| After enzyme digestion the plasmid | 1 μL |
| vector of pSpCas9(BB)-2A-GFP | |
| T4 Ligase (NEB) | 1 μL |
| 10X T4 Ligase Buffer (NEB) | 2 μL |
| ddH₂O | up to 20 μL |

9. Formation Experiment of White to Blue Clone

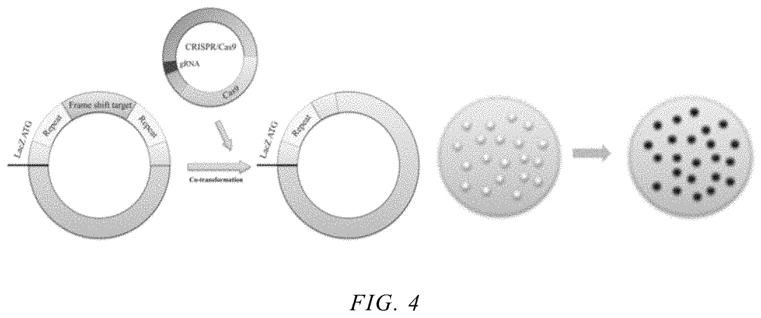

When the pCas9 plasmid contained the sgRNA sequence and the pMD-repeat plasmid contained the corresponding sgRNA target sequence co-transform DH5α competent cells, the Cas9 enzyme will recognize and cut the target sequence under the sgRNA mediation, causing DNA double-strand breaks. Homologous recombination would occur between repeated segments. Only one repeat sequence will have the reading frame of the lacZ gene. It would change from a frameshift state to a non-frameshift state. Under the induction of X-gal and IPTG, blue colonies would form. Experiment The schematic diagram was shown in FIG. 4.

Co-Transformation Experiment

When the sequence contained the pCas9 plasmid of sgRNA and the pMD-repeat plasmid contained the corresponding rake sequence of the sgRNA were transformed into 50 μL DH5α competent cells), 800 μL LB liquid medium, at 37° C. for 40 min. Cultured on a plate contained X-gal and TPTG-chloramphenicol-ampicillin at 37° C. and observed the ratio of blue colonies to the total colonies. Pick the blue colonies and placed them in LB liquid contained chloramphenicol-ampicillin resistance After 12 hours of incubation in the medium, sequencing with the universal primer of pMD-19T was performed to observe whether the target sequence was digested by enzymes and homologous recombination between the repeated sequences occurred.

10. The Experiment Results of White to Blue Clone Formation

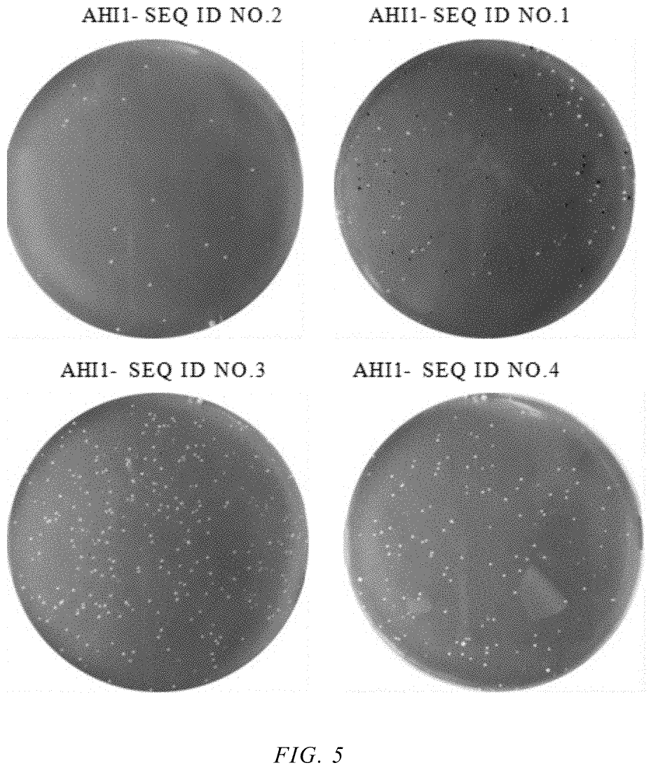

The pCas9 plasmid loaded with AHI1 sgRNA and the corresponding pMD-repeat plasmid loaded with the target sequence were respectively co-transformed into DH5α competent cells in equal amounts, cultured on X-gal-IPTG-Cl-Amp plates, and repeated three times. Representative colonies the growth chart was shown in FIG. 5. According to the colony map of the four sgRNAs of AHI1, it could be found that the s gRNA (SEQ ID NO: 1) of the present invention has a higher percentage of cyanobacteria (61%) compared with the sgRNA (SEQ ID NO: 2, SEQ ID NO: 3, SEQ ID NO: 4)

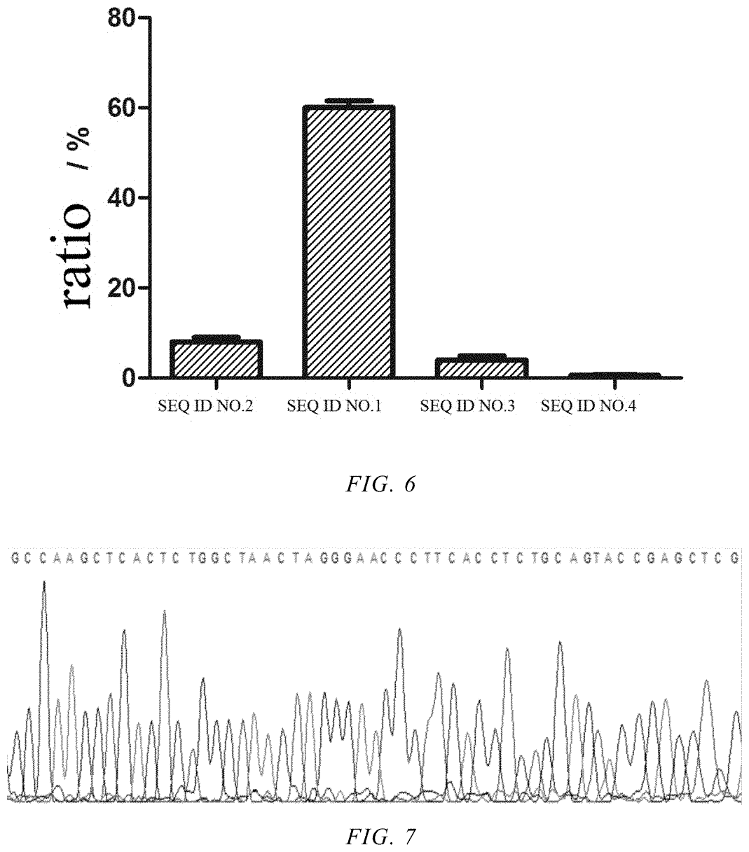

The ratio of cyanobacteria to total colonies was low (about 8%, about 4%, <1%). As shown in FIG. 6, the editing efficiency of the sgRNA of the present invention was high. In each plate, picked the blue colonies and cultured them with LB liquid medium contained Cl-Amp, sended the bacterial liquid for sequencing, and the diagram of pMD-repeat plasmid repair sequencing were consistent, which were shown in FIG. 7.

In the formation experiment of white to blue clone, the replacement of the partial sequence of the lacZ gene of the pMD-19T plasmid destroyed the f-gal reading frame, which could not be produced when transformed alone β-gal, showing white colonies; when corresponded to the target sequence) When the pCas9 plasmid of the sgRNA sequence was co-transformed, the Cas9 enzyme would cut the corresponding target sequence under the guidance of the sgRNA to form a DNA double-strand break (DSB). Read frame, blue colonies were formed under the induction of X-gal and IPTG. The proportion of blue colonies in all colonies reflects the editing activity of the sgRNA. If the editing activity of the sgRNA was low, the pMD-19T plasmid with the target sequence digested was less. The ratio of white colonies to the total colonies in the colonies was high. It was can be seen from the above, the sgRNA disclosed by the present invention had excellent editing efficiency and had achieved unexpected technical effects.

1. An sgRNA for an AHI1 gene, comprising a nucleotide sequence set forth in SEQ ID NO: 1.

2. The sgRNA for AHI1 gene according to claim 1, wherein an editing efficiency of the sgRNA of the AHI1 gene is 60% to 62%.

3. A pharmaceutical composition comprising the sgRNA of claim 1.

4. The pharmaceutical composition according to claim 3, further comprising a drug carrier.

5. The pharmaceutical composition according to claim 4, wherein the drug carrier is a polymer-support or a cell carrier.

6. A plasmid of an AHI1 gene, comprising a carrier and the sgRNA according to claim 1.

7. The plasmid of the AHI1 gene according to claim 6, wherein the carrier is a pCas9 plasmid.

8. A method comprising: applying the sgRNA according to claim 1 in preparing a pharmaceutical composition.

9. The method according to claim 8, wherein an editing efficiency of the sgRNA of an AHI1 gene is 60% to 62%.

10. The method according to claim 8, wherein the pharmaceutical composition treats Joubert syndrome.


SEQUENCE LISTING (28 sequences)

SEQ ID NO: 1 - DNA, 23 bp, Artificial Sequence
  sgRNA
gataatgtctccgcgatggatgg

SEQ ID NO: 2 - DNA, 23 bp, Artificial Sequence
  sgRNA
ctcggataatgtctccgcgatgg

SEQ ID NO: 3 - DNA, 23 bp, Artificial Sequence
  sgRNA
aattggatatccatcccggctgg

SEQ ID NO: 4 - DNA, 23 bp, Artificial Sequence
  sgRNA
gatgaactaaccatccatcgcgg

SEQ ID NO: 5 - DNA, 25 bp, Artificial Sequence
  sgRNA
aaacctcggataatgtctccgcgag

SEQ ID NO: 6 - DNA, 25 bp, Artificial Sequence
  sgRNA
aaaactcgcggagacattatccgag

SEQ ID NO: 7 - DNA, 25 bp, Artificial Sequence
  sgRNA
aaacgataatgtctccgcgatggag

SEQ ID NO: 8 - DNA, 25 bp, Artificial Sequence
  sgRNA
aaaactccatcgcggagacattatc

SEQ ID NO: 9 - DNA, 25 bp, Artificial Sequence
  sgRNA
aaacaattggatatccatcccggcg

SEQ ID NO: 10 - DNA, 25 bp, Artificial Sequence
  sgRNA
aaaacgccgggatggatatccaatt

SEQ ID NO: 11 - DNA, 24 bp, Artificial Sequence
  sgRNA
aaacgatgaactaaccatccatcg

SEQ ID NO: 12 - DNA, 24 bp, Artificial Sequence
  sgRNA
aaaacgatggatggttagttcatc

SEQ ID NO: 13 - DNA, 34 bp, Artificial Sequence
  sgRNA
agcttactcggataatgtctccgcgatggggtac

SEQ ID NO: 14 - DNA, 26 bp, Artificial Sequence
  sgRNA
cccatcgcggagacattatccgagta

SEQ ID NO: 15 - DNA, 34 bp, Artificial Sequence
  sgRNA
agcttggataatgtctccgcgatggatggggtac

SEQ ID NO: 16 - DNA, 26 bp, Artificial Sequence
  sgRNA
cccatccatcgcggagacattatcca

SEQ ID NO: 17 - DNA, 34 bp, Artificial Sequence
  sgRNA
agctttaattggatatccatcccggctggggtac

SEQ ID NO: 18 - DNA, 26 bp, Artificial Sequence
  sgRNA
cccagccgggatggatatccaattaa

SEQ ID NO: 19 - DNA, 34 bp, Artificial Sequence
  sgRNA
agcttagatgaactaaccatccatcgcggggtac

SEQ ID NO: 20 - DNA, 26 bp, Artificial Sequence
  sgRNA
cccgcgatggatggttagttcatcta

SEQ ID NO: 21 - DNA, 25 bp, Artificial Sequence
  sgRNA
caccgctcggataatgtctccgcga

SEQ ID NO: 22 - DNA, 25 bp, Artificial Sequence
  sgRNA
aaactcgcggagacattatccgagc

SEQ ID NO: 23 - DNA, 24 bp, Artificial Sequence
  sgRNA
caccgataatgtctccgcgatgga

SEQ ID NO: 24 - DNA, 24 bp, Artificial Sequence
  sgRNA
aaactccatcgcggagacattatc

SEQ ID NO: 25 - DNA, 25 bp, Artificial Sequence
  sgRNA
caccgaattggatatccatcccggc

SEQ ID NO: 26 - DNA, 25 bp, Artificial Sequence
  sgRNA
aaacgccgggatggatatccaattc

SEQ ID NO: 27 - DNA, 24 bp, Artificial Sequence
  sgRNA
caccgatgaactaaccatccatcg

SEQ ID NO: 28 - DNA, 24 bp, Artificial Sequence
  sgRNA
aaaccgatggatggttagttcatc